(19) United States
(12) Patent Application Publication
(10) Pub. No.: US 2020/0089961 A1
(43) Pub. Date: Mar. 19, 2020

(54) METHOD FOR EVALUATING SOCIAL INTELLIGENCE AND APPARATUS USING THE SAME

(71) Applicant: ELECTRONICS AND TELECOMMUNICATIONS RESEARCH INSTITUTE, Daejeon (KR)

(72) Inventors: Mi-Young CHO, Daejeon (KR); Jae-Hong KIM, Daejeon (KR); Jae-Yeon LEE, Daejeon (KR)

(21) Appl. No.: 16/213,857

(22) Filed: Dec. 7, 2018

(30) Foreign Application Priority Data
Sep. 17, 2018 (KR) 10-2018-0111127

(51) Int. Cl. G06K 9/00; A61B 5/16

(52) U.S. Cl. CPC G06K 9/00744; A61B 5/167; G06K 9/00335

(57) ABSTRACT

Disclosed herein are a method for evaluating social intelligence and an apparatus for the same. The method includes creating multiple segmented video clips by segmenting, based on behavior recognition, an observation video sequence that captures the social interaction behavior of the target to be evaluated; and evaluating the social intelligence of the target by calculating an evaluation score based on the similarities between ground truth, created based on social interaction analysis, and the multiple segmented video clips.

### CROSS REFERENCE TO RELATED APPLICATION

This application claims the benefit of Korean Patent Application No. 10-2018-0111127, filed Sep. 17, 2018, which is hereby incorporated by reference in its entirety into this application.

### BACKGROUND OF THE INVENTION

### 1. Technical Field

The present invention relates generally to technology for evaluating human social intelligence, and more particularly to technology for automatically evaluating a social intelligence level pertaining to social interaction behavior between people captured on video.

### 2. Description of the Related Art

Conventional human intelligence tests focus on classification of the targets to be evaluated into grades for a specific purpose and on anticipation of their performance, and mainly deal with, for example, school records and the like. This trend is connected with a narrow concept of intelligence measurement based on IQ. Here, when considering various human activities, such intelligence measurements are of limited usefulness in evaluating overall human intelligence because a very limited range of abilities can be evaluated.

Recently, as alternatives to such an intelligence concept, theories and methods overcoming the narrow concept of intelligence measurements have been proposed. Recent intelligence research includes not only school records but also creativity, social skills, artistic talent, appraisal and expression of emotion, morality, personality, motivation, and the like in intelligence concepts. Particularly, Howard Gardner has pointed out that the traditional intelligence system emphasizes only linguistic and logical-mathematical abilities and has suggested a theory of multiple kinds of intelligence that include musical intelligence, linguistic intelligence, logical-mathematical intelligence, spatial intelligence, bodily-kinesthetic intelligence, interpersonal intelligence, intrapersonal intelligence, and naturalistic intelligence in consideration of various kinds of human intelligence. Here, interpersonal intelligence, otherwise known as social intelligence, is defined as the ability to sensitively pay attention to social relationships using social knowledge in a social context, to easily adapt to new social situations, and to flexibly process information in order to effectively solve social problems in daily life.

The cognitive intelligence of humans may be evaluated using evaluation methods and measurement tools, such as IQ tests, dementia screening tests, children's intelligence tests, and the like. Most of these methods and measurement tools are traditional cognitive neuropsychological assessment tools and are based on self-reporting methods, rather than on observation. Conversely, social intelligence is evaluated based on observation and aims at evaluating how well individuals socially interact with others using a standardized evaluation tool known as Evaluation of Social Interaction (ESI). However, ESI is capable of being performed only by observation by highly skilled experts, and thus faces a limitation in that there is a shortage of specialists and in that it is time-consuming.

### DOCUMENTS OF RELATED ART

(Patent Document 1) Korean Patent Application Publication No. 10-2013-0046200, published on May 7, 2013 and titled “Social ability training apparatus and method thereof”.

### SUMMARY OF THE INVENTION

An object of the present invention is to apply video data analysis and comparison methods to the evaluation of human social intelligence.

Another object of the present invention is to provide an automated evaluation tool for evaluating human social intelligence levels based on standardized evaluation criteria.

A further object of the present invention is to identify, in advance, people who have a social intelligence problem and to provide proper treatment and care services.

Yet another object of the present invention is to provide a method that enables laymen to evaluate human social intelligence in a short time more effectively than when using a method in which highly skilled experts observe an evaluation target for a long time and acquire a result therefrom.

Still another object of the present invention is to provide a method for evaluating social intelligence through which the development of social intelligence of an evaluation target may be continuously evaluated and tracked regardless of the location of the evaluation target.

In order to accomplish the above objects, a method for evaluating social intelligence according to the present invention includes segmenting, based on behavior recognition, an observation video sequence that captures the social interaction behavior of the target to be evaluated, thereby creating multiple segmented video clips; and calculating an evaluation score based on similarities between ground truth, which is created based on social interaction analysis, and the multiple segmented video clips, thereby evaluating the social intelligence of the target.

Here, the ground truth may correspond to multiple verification video clips that are created by classifying an input video sequence pertaining to social interaction based on specific behavior items of an Evaluation of Social Interaction (ESI) scenario.

Here, evaluating the social intelligence of the target may be configured to calculate the evaluation score by applying a score for each ESI item and a weight for specific behavior to each of the similarities, the score for each ESI item being set based on the ESI scenario, and the weight for specific behavior being set based on the specific behavior items.

Here, evaluating the social intelligence of the target may include sequentially comparing the multiple segmented video clips with the multiple verification video clips and measuring the similarities through comparison of the content of the video clips and comparison of the context of the content that precedes and follows the video clips.

Here, the similarities may be measured using cosine similarity between feature information extracted from the multiple segmented video clips and feature information extracted from the multiple verification video clips.

Here, the feature information may be behavior recognition information and facial expression recognition information, which are extracted from image data, and conversation information and emotion recognition information, which are extracted from sound data.

Here, creating the multiple segmented video clips may be configured to segment the observation video sequence into the multiple segmented video clips by performing behavior recognition based on at least one of an object detection function, an object-tracking function, and a gesture recognition function.

Also, an apparatus for evaluating social intelligence according to an embodiment of the present invention includes a processor for creating multiple segmented video clips by segmenting, based on behavior recognition, an observation video sequence that captures the social interaction behavior of the target to be evaluated, for calculating an evaluation score based on similarities between ground truth, which is created based on social interaction analysis, and the multiple segmented video clips, and for evaluating the social intelligence of the target; and memory for storing the ground truth.

Here, the ground truth may correspond to multiple verification video clips that are created by classifying an input video sequence pertaining to social interaction based on specific behavior items of an Evaluation of Social Interaction (ESI) scenario.

Here, the processor may calculate the evaluation score by applying a score for each ESI item and a weight for specific behavior to each of the similarities, the score for each ESI item being set based on the ESI scenario, and the weight for specific behavior being set based on the specific behavior items.

Here, the processor may sequentially compare the multiple segmented video clips with the multiple verification video clips and may measure the similarities through comparison of the content of the video clips and comparison of the context of the content that precedes and follows the video clips.

Here, the similarities may be measured using cosine similarity between feature information extracted from the multiple segmented video clips and feature information extracted from the multiple verification video clips.

Here, the feature information may be behavior recognition information and facial expression recognition information, which are extracted from image data, and conversation information and emotion recognition information, which are extracted from sound data.

Here, the processor may segment the observation video sequence into the multiple segmented video clips by performing behavior recognition based on at least one of an object detection function, an object-tracking function, and a gesture recognition function.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a view that shows the configuration of a social intelligence evaluation process according to an embodiment of the present invention;

FIG. 2 is a flowchart that shows a method for evaluating social intelligence according to an embodiment of the present invention;

FIG. 3 is a view that shows an example of a video clip according to the present invention;

FIG. 4 is a view that shows an example of the configuration of social interaction;

FIG. 5 is a view that shows an example of the process of segmenting a video clip according to the present invention;

FIG. 6 is a view that shows an example of ground truth according to the present invention;

FIG. 7 is a view that shows an example of the process of measuring cosine similarity according to the present invention;

FIG. 8 is a view that shows an example of feature information based on image data and sound data of a video clip according to the present invention;

FIG. 9 is a view that shows the specific configuration of a social intelligence evaluation process according to the present invention;

FIG. 10 is a block diagram that shows an apparatus for evaluating social intelligence according to an embodiment of the present invention; and

FIG. 11 is a view that shows a computer system according to an embodiment of the present invention.

### DESCRIPTION OF THE PREFERRED EMBODIMENTS

The present invention will be described in detail below with reference to the accompanying drawings. Repeated descriptions and descriptions of known functions and configurations which have been deemed to unnecessarily obscure the gist of the present invention will be omitted below. The embodiments of the present invention are intended to fully describe the present invention to a person having ordinary knowledge in the art to which the present invention pertains. Accordingly, the shapes, sizes, etc. of components in the drawings may be exaggerated in order to make the description clearer.

Hereinafter, a preferred embodiment of the present invention will be described in detail with reference to the accompanying drawings.

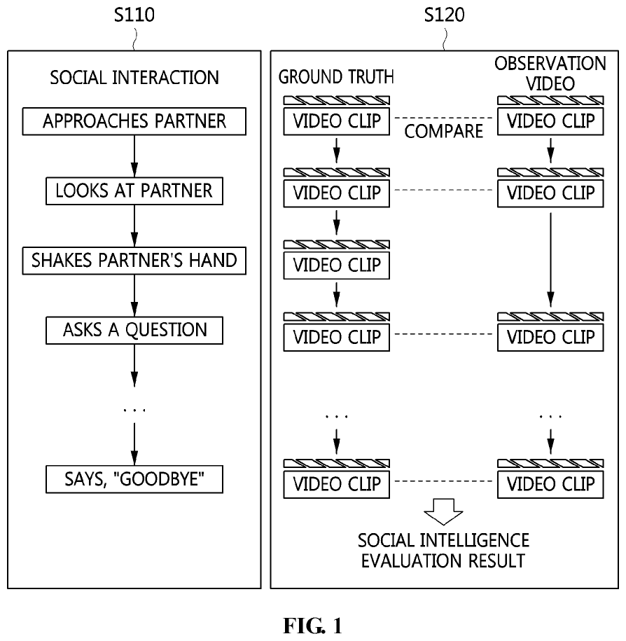

FIG. 1 is a view that shows the configuration of a social intelligence evaluation process according to an embodiment of the present invention.

Referring to FIG. 1, social intelligence evaluation according to an embodiment of the present invention may be performed by classifying social interaction as specific behavior at step S110 and by measuring the similarity between two pieces of video data at step S120.

Here, step S110 may be the step of classifying social interaction to be evaluated as specific behavior.

For example, social interaction may include multiple actions, but the seven of most essential types of social interaction, which are shown in Table 1 and are classified with reference to Evaluation of Social Interaction (ESI) scenarios, may be used to evaluate social intelligence.

TABLE 1

| category | subcategory |
|---|---|
| collecting | collecting information about favorite books from friends |
| information from | collecting information about mobile phone functions |
| others | collecting information from a job applicant during an interview |
| sharing information | sharing information about what to order with friends in a restaurant |
| with others | sharing information about artwork |
| | sharing a training course with colleagues |
| solving a problem | planning to rearrange living room furnishing |
| or making a | selecting a book to read for the next book club meeting |
| decision | selecting a part to complete in an art project |
| collaboration and | cooking together |
| production | working together on making a collage |
| | doing homework together |
| acquiring a | interacting with someone with regard to a bank account or a |
| product or a | postal order |
| service | interacting with someone with regard to the purchase of a |
| | movie or theater ticket |
| | requesting the support of a service provider |
| providing a | interacting with someone to order food in a restaurant |
| product or a | interacting with someone with regard to the sale of a ticket |
| service | |
| participating in | bantering with others while drinking coffee or having a meal |
| social | bantering with others while waiting for a bus |
| conversation and | bantering with a hair designer while getting a haircut |
| small talk | |

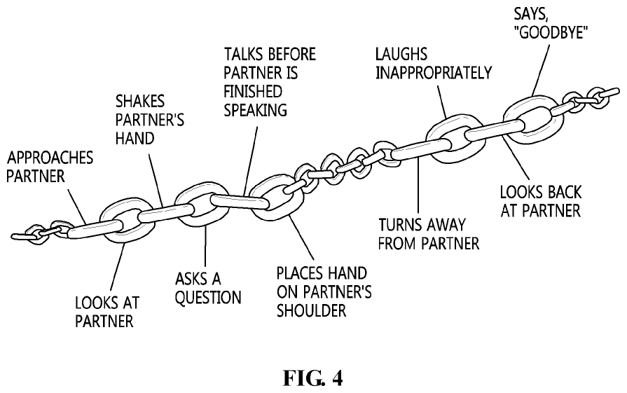

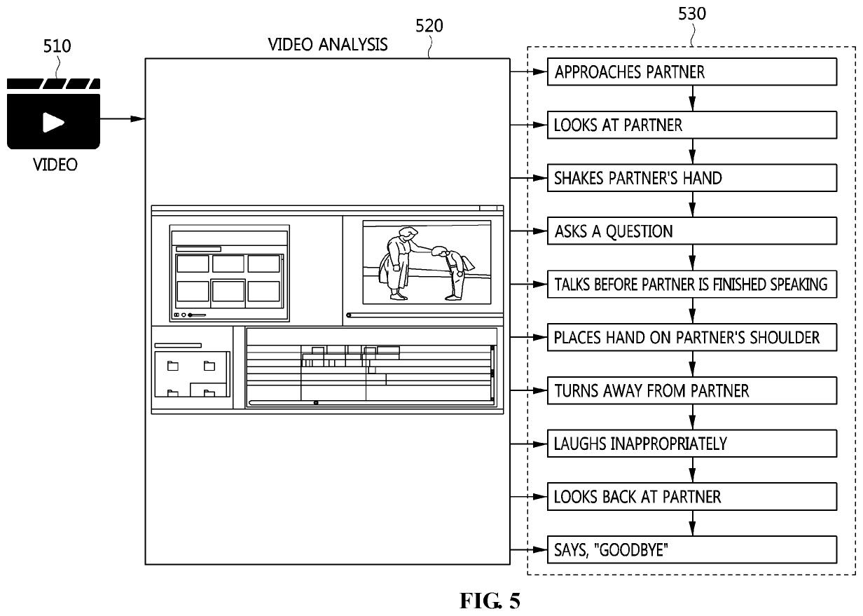

Here, human social interaction may be considered to be configured as a chain of small and observable social behaviors, as shown in FIG. 4. Accordingly, when such a small and observable social behavior is defined as a single meaningful video clip in a video sequence, the overall social interaction may correspond to a set of video clips in which multiple video clips are sequentially arranged.

Accordingly, the present invention segments a video sequence, corresponding to social interaction, into small video clips, each of which corresponds to specific behavior, based on a video analysis process, thereby creating ground truth for understanding and interpreting the story included in the video sequence.

Then, at step S120, ground truth and observation video, which captures the target to be evaluated, are compared with each other in units of video clips, and the similarity therebetween is measured, whereby the social intelligence of the target may be evaluated based on the measured similarity.

Here, the observation video, which captures the social interaction of the target to be evaluated, is segmented into video clips, and the video clips may be compared with video clips included in the ground truth.

Here, the similarity between the ground truth and the observation video may be measured using cosine similarity between feature information extracted from the image data and the sound data included in the video clips of the ground truth and that of the observation video. That is, feature vectors extracted from image data and sound data may be used as input values for measuring the cosine similarity.

For example, feature vectors may be extracted from image data using human detection and tracking methods, behavior and gesture recognition methods, and facial expression recognition methods. Also, feature vectors may be extracted from sound data using conversation recognition methods, background noise cancellation methods, and emotion recognition methods.

Here, the similarity between the ground truth and the observation video may be measured in consideration of the context of the content that precedes and follows the video clips as well as the scenes of the corresponding video clips, and social intelligence may be evaluated based thereon.

Through the above-described configuration of a social intelligence evaluation process according to an embodiment of the present invention, laymen may evaluate human social intelligence in a short time, compared to the method in which high skilled experts observe the target to be evaluated for a long time and acquire a result therefrom. Also, as long as video that captures social interaction is available, social intelligence may be evaluated anywhere. Accordingly, it is possible to continuously observe and track the development process of a target to be evaluated.

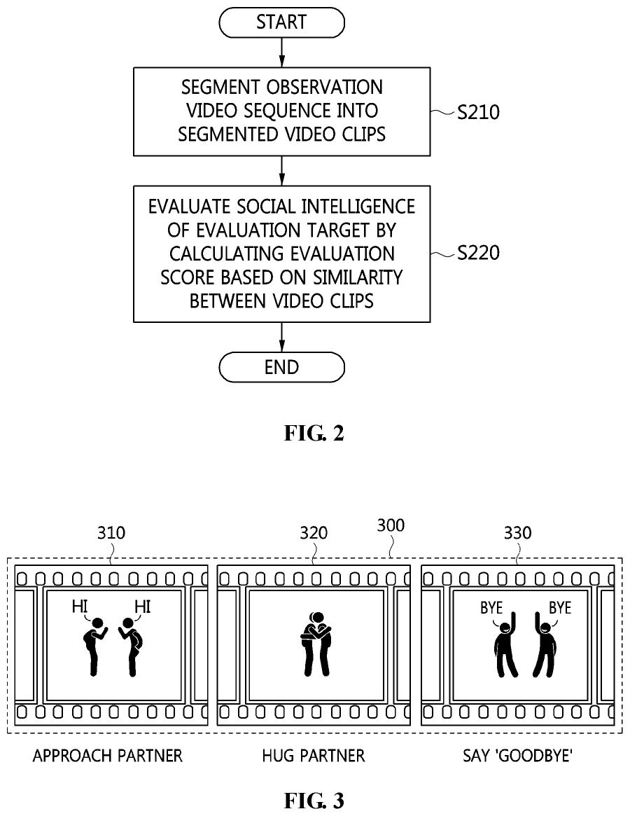

FIG. 2 is a flowchart that shows a method for evaluating social intelligence according to an embodiment of the present invention.

Generally, as opposed to cognitive intelligence evaluation based on a self-reporting method, social intelligence evaluation may be performed by observers. Accordingly, social intelligence evaluation requires highly skilled experts, but there is a lack of people who are specialized therein, and the evaluation process is time-consuming. Therefore, it is difficult to evaluate human social intelligence.

Accordingly, the present invention applies video data analysis and comparison methods to the evaluation of human social intelligence, thereby providing a method in which whether social interaction behavior between people shown in video is appropriate is automatically determined and evaluated.

Referring to FIG. 2, in the method for evaluating social intelligence according to an embodiment of the present invention, an observation video sequence that captures the social interaction behavior of the target to be evaluated is segmented based on behavior recognition, whereby multiple segmented video clips are created at step S210.

For example, a single long observation video sequence 300 may be segmented, whereby multiple segmented video clips 310 to 330 may be created, as shown in FIG. 3. Here, the content of the observation video sequence 300 is analyzed, and the behavior of the target to be evaluated, which is captured in the observation video sequence 300, may be sequentially recognized as behavior corresponding to ‘approaches the partner’, ‘hugs the partner’, and ‘says goodbye’. Accordingly, the observation video sequence 300 is segmented into parts, each of which corresponds to a specific behavior, and the segmented parts may be created as a segmented video clip 310 corresponding to ‘approaches the partner’, a segmented video clip 320 corresponding to ‘hugs the partner’, and a segmented video clip 330 corresponding to ‘says goodbye’.

Here, behavior recognition is performed based on at least one of an object detection function, an object-tracking function, and a gesture recognition function, whereby the observation video sequence may be segmented into multiple segmented video clips.

For example, the target to be evaluated may be detected from the observation video sequence through object detection and tracking, and the behavior of the target is recognized through gesture recognition, whereby the point at which the video sequence is segmented may be determined.

Also, in the method for evaluating social intelligence according to an embodiment of the present invention, the social intelligence of the target is evaluated at step S220 by calculating an evaluation score based on the similarities between the ground truth, which is created based on social interaction analysis, and the multiple segmented video clips.

Here, human social interaction may be considered to be configured as a chain of small and observable social behaviors, as shown in FIG. 4. Accordingly, when such a small and observable social behavior is defined as a single meaningful video clip in the input video sequence, the overall social interaction may be regarded as a set of video clips in which multiple video clips are sequentially arranged.

Therefore, the ground truth created based on social interaction analysis may also be such a set of video clips.

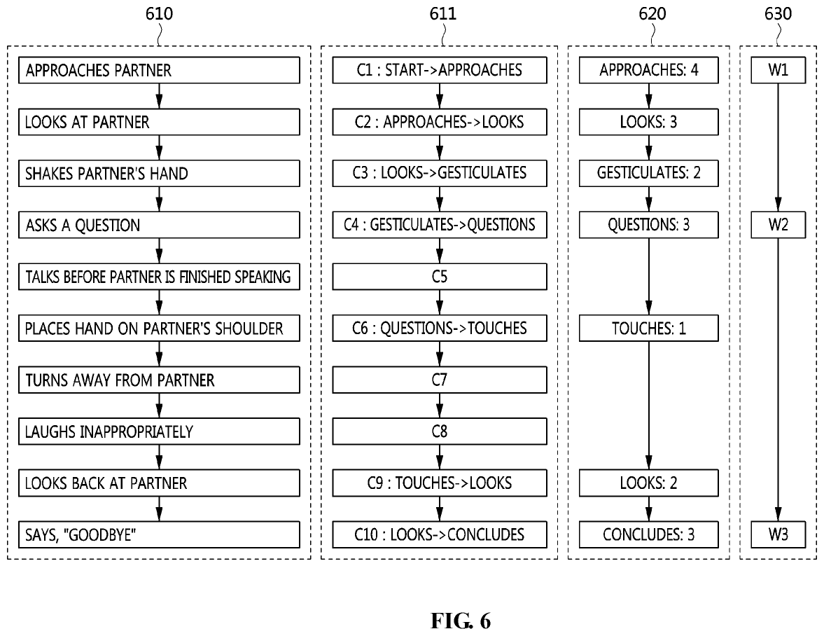

Referring to FIG. 5 and FIG. 6, the process of creating ground truth is as follows. First, when a video sequence 510 pertaining to social interaction is input as shown in FIG. 5, a video analysis module 520 classifies social interaction as specific behavior through preprocessing, whereby the video sequence 510 may be segmented into verification video clips 530. Then, an expert who specializes in social intelligence evaluation may set a score 620 for each ESI item pertaining to each verification video clip 610 and a weight 630 for each video clip, that is, a weight for specific behavior in social interaction, as shown in FIG. 6. Then, ground truth for understanding and interpreting the story of video data may be created based on the contextual information 611 that precedes and follows each video clip.

That is, the ground truth according to the present invention may be multiple verification video clips that are created by segmenting an input video sequence pertaining to social interaction into specific behavior items that are classified with reference to an Evaluation of Social Interaction (ESI) scenario.

Here, the multiple verification video clips may include image data and sound data. Accordingly, the ground truth may include behavior recognition information, gesture recognition information, facial expression recognition information, and the like based on the image data, and may include conversation information, background noise information, emotion recognition information, and the like based on the sound data.

Here, a score for each ESI item, which is set based on the ESI scenario, and a weight for specific behavior, which is set based on the classified specific behavior items, are applied to the similarity, whereby an evaluation score may be calculated.

For example, an evaluation score may be assumed to be calculated based on the multiple segmented video clips 310 to 330 that are acquired from the observation video sequence 300 shown in FIG. 3. First, when similarity is measured by comparing ground truth with the segmented video clip 310, corresponding to ‘approaches the partner’, an evaluation score for the segmented video clip 310 may be calculated by applying an ESI item score, corresponding to the segmented video clip 310, and a weight corresponding thereto to the similarity. Similar to this, an evaluation score for the segmented video clip 320, corresponding to ‘hugs the partner’ and an evaluation score for the segmented video clip 330, corresponding to ‘says goodbye’, are calculated, and then all of these scores are added, whereby the final evaluation score may be calculated.

The above example is merely an embodiment, and the evaluation score may be calculated based on similarity, a score for each ESI item, and a weight for specific behavior, but the method by which to use these three factors in order to calculate the evaluation score is not limited to any specific method.

Here, the multiple segmented video clips are sequentially compared with the multiple verification video clips, in which case similarities may be measured not only by comparing the content of the video clips but also by comparing the context of the content that precedes and follows the video clips. That is, when multiple video clips are used to evaluate the social intelligence of the target to be evaluated, similarity to the ground truth is measured in consideration of the context of the content preceding and following the video clip, rather than using only the scene included in the corresponding video clip, whereby social intelligence may be evaluated based thereon.

Here, the similarity may be measured using the cosine similarity between feature information extracted from the multiple segmented video clips and that extracted from the multiple verification video clips.

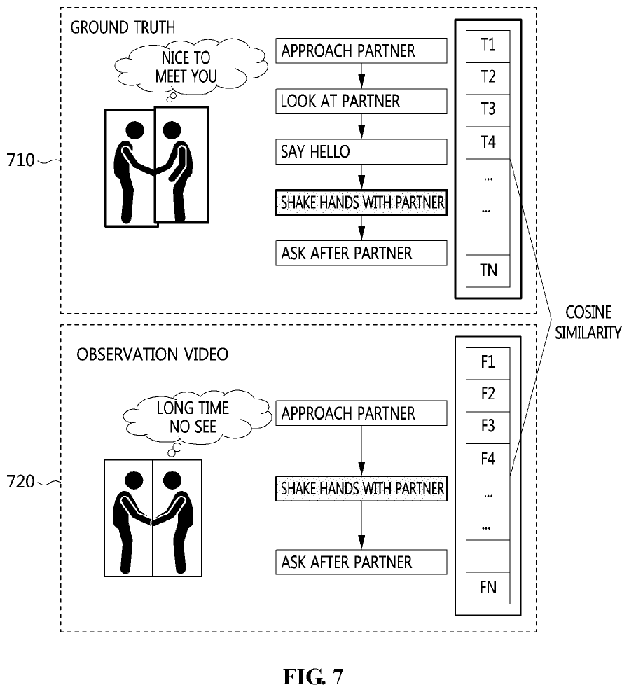

For example, referring to FIG. 7, similarities are measured by comparing the multiple verification video clips included in the ground truth 710 with the multiple segmented video clips included in an observation video sequence 720. Here, the similarity between any two video clips may be measured using cosine similarity between pieces of feature information extracted from image data and sound data. Here, feature vectors that correspond to the pieces of feature information extracted from the image data and the sound data may be used as input values for measuring the cosine similarity between the two video clips.

Here, the feature information may be behavior recognition information and facial expression recognition information extracted from the image data and conversation information and emotion recognition information extracted from the sound data.

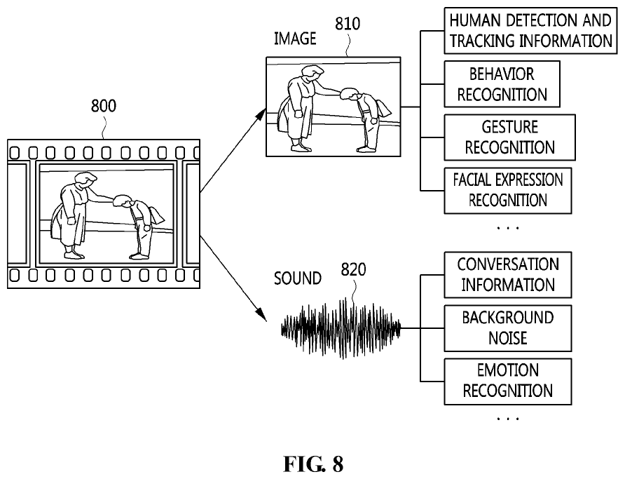

For example, referring to FIG. 8, feature vectors related to human detection and tracking information, behavior recognition information, gesture recognition information, and facial expression recognition information may be extracted from the image data of the video clip 800 and used to measure the similarity. Also, feature vectors related to conversation information, background noise information, and emotion recognition information may be extracted from the sound data 820 of the video clip 800 and used to measure the similarity.

Also, although not illustrated in FIG. 2, in the method for evaluating social intelligence according to an embodiment of the present invention, various kinds of information generated during the above-described process of evaluating social intelligence according to an embodiment of the present invention may be stored in a separate storage module.

Through the above-described method for evaluating social intelligence, laymen may also evaluate human social intelligence more effectively than when using a method in which highly skilled experts observe the target to be evaluated for a long time and acquire a result therefrom.

Also, it is possible to continuously observe and track the development process of the target to be evaluated regardless of where the target is.

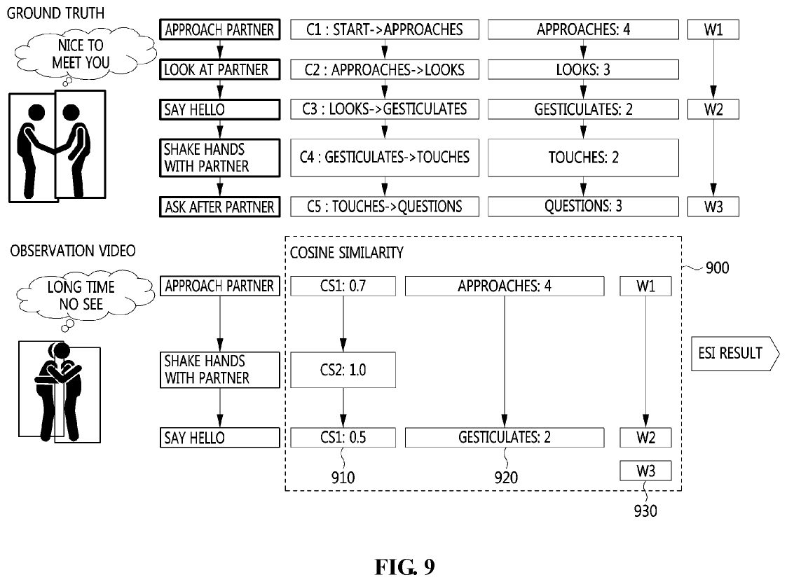

FIG. 9 is a view that shows the specific configuration of a social intelligence evaluation process according to the present invention.

Referring to FIG. 9, social intelligence evaluation according to the present invention may be performed by defining ground truth for social intelligence evaluation based on the process of classifying social interaction as specific behavior and by comparing the ground truth with observation video that captures the social interaction behavior of the target to be evaluated.

Here, when verification video clips included in the ground truth are compared with segmented video clips created by segmenting the observation video, image data interpretation information, sound data interpretation information, time information, contextual information, and the like may be compared.

The evaluation result 900 generated through the comparison may include not only the result 910 acquired by measuring the similarity between two pieces of data but also a score 920 for each ESI item and a weight 930 for specific behavior, which are applied to the similarity result 910.

Therefore, the evaluation score for the social interaction behavior of the target to be evaluated may be calculated based on the factors included in the evaluation result 900, and the social intelligence of the target may be evaluated based on the evaluation score.

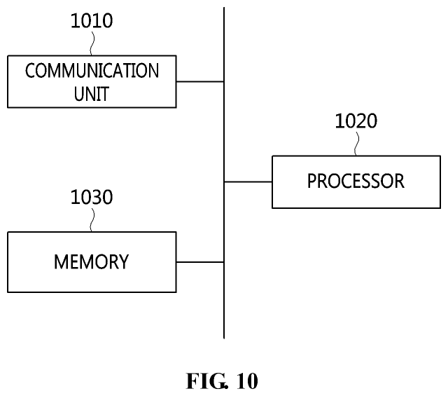

FIG. 10 is a block diagram that shows an apparatus for evaluating social intelligence according to an embodiment of the present invention.

Referring to FIG. 10, the apparatus for evaluating social intelligence according to an embodiment of the present invention includes a communication unit 1010, a processor 1020, and memory 1030.

The communication unit 1010 functions to send and receive information that is necessary in order to evaluate the social intelligence of the target to be evaluated through a communication network. Particularly, the communication unit 1010 according to an embodiment of the present invention may receive an observation video sequence that captures the target to be evaluated, or may provide an evaluation result to the target to be evaluated.

The processor 1020 creates multiple segmented video clips by segmenting the observation video sequence that captures the social interaction behavior of the target to be evaluated based on behavior recognition.

For example, a single long observation video sequence 300 may be segmented, whereby multiple segmented video clips 310 to 330 may be created, as shown in FIG. 3. Here, the content of the observation video sequence 300 is analyzed, and the behavior of the target to be evaluated, which is captured in the observation video sequence 300, may be sequentially recognized as behavior corresponding to ‘approaches the partner’, ‘hugs the partner’, and ‘says goodbye’. Accordingly, the observation video sequence 300 is segmented into parts, each of which corresponds to a specific behavior, and the segmented parts may be created as a segmented video clip 310 corresponding to ‘approaches the partner’, a segmented video clip 320 corresponding to ‘hugs the partner’, and a segmented video clip 330 corresponding to ‘says goodbye’.

Here, behavior recognition is performed based on at least one of an object detection function, an object-tracking function, and a gesture recognition function, whereby the observation video sequence may be segmented into multiple segmented video clips.

For example, the target to be evaluated may be detected from the observation video sequence through object detection and tracking, and the behavior of the target may be recognized through gesture recognition, whereby the point at which the video sequence is segmented may be determined.

Also, the processor 1020 calculates an evaluation score based on the similarities between the ground truth, which is created based on social interaction analysis, and the multiple segmented video clips, thereby evaluating the social intelligence of the target to be evaluated.

Here, human social interaction may be considered to be configured as a chain of small and observable social behaviors, as shown in FIG. 4. Accordingly, when such a small and observable social behavior is defined as a single meaningful video clip in the input video sequence, the overall social interaction may be regarded as a set of video clips in which multiple video clips are sequentially arranged.

Therefore, the ground truth created based on social interaction analysis may also be such a set of video clips.

Referring to FIG. 5 and FIG. 6, the process of creating ground truth is as follows. First, when a video sequence 510 pertaining to social interaction is input as shown in FIG. 5, a video analysis module 520 classifies social interaction as specific behavior through preprocessing, whereby the video sequence 510 may be segmented into verification video clips 530. Then, an expert who specializes in social intelligence evaluation may set a score 620 for each ESI item pertaining to each verification video clip 610 and a weight 630 for each video clip, that is, a weight for specific behavior in social interaction, as shown in FIG. 6. Then, ground truth for understanding and interpreting the story of video data may be created based on the contextual information 611 that precedes and follows each video clip.

That is, the ground truth according to the present invention may be multiple verification video clips that are created by segmenting an input video sequence pertaining to social interaction into specific behavior items that are classified with reference to an Evaluation of Social Interaction (ESI) scenario.

Here, the multiple verification video clips may include image data and sound data. Accordingly, the ground truth may include behavior recognition information, gesture recognition information, facial expression recognition information, and the like based on the image data, and may include conversation information, background noise information, emotion recognition information, and the like based on the sound data.

Here, a score for each ESI item, which is set based on the ESI scenario, and a weight for specific behavior, which is set based on the classified specific behavior items, are applied to the similarity, whereby an evaluation score may be calculated.

For example, an evaluation score may be assumed to be calculated based on the multiple segmented video clips 310 to 330 that are acquired from the observation video sequence 300 shown in FIG. 3. First, when similarity is measured by comparing ground truth with the segmented video clip 310, corresponding to ‘approaches the partner’, an evaluation score for the segmented video clip 310 may be calculated by applying an ESI item score corresponding to the segmented video clip 310 and a weight corresponding thereto to the similarity. Similar to this, an evaluation score for the segmented video clip 320, corresponding to ‘hugs the partner’, and an evaluation score for the segmented video clip 330, corresponding to ‘says goodbye’, are calculated, and then all of these scores are added, whereby the final evaluation score may be calculated.

The above example is merely an embodiment, in which the evaluation score is calculated based on similarity, a score for each ESI item, and a weight for specific behavior, but the method by which to use these three factors in order to calculate the evaluation score is not limited to any specific method.

Here, the multiple segmented video clips are sequentially compared with the multiple verification video clips, in which case similarities may be measured not only by comparing the content of the video clips but also by comparing the context of the content that precedes and follows the video clips. That is, when multiple video clips are used to evaluate the social intelligence of the target to be evaluated, similarity to the ground truth is measured in consideration of the context of the content preceding and following the video clip, rather than using only the scene included in the corresponding video clip, whereby social intelligence may be evaluated based thereon.

Here, the similarity may be measured using the cosine similarity between feature information extracted from the multiple segmented video clips and that extracted from the multiple verification video clips.

For example, referring to FIG. 7, similarities are measured by comparing the multiple verification video clips included in the ground truth 710 with the multiple segmented video clips included in an observation video sequence 720. Here, the similarity between any two video clips may be measured using cosine similarity between pieces of feature information extracted from image data and sound data. Here, feature vectors that correspond to the pieces of feature information extracted from the image data and the sound data may be used as input values for measuring the cosine similarity between the two video clips.

Here, the feature information may be behavior recognition information and facial expression recognition information extracted from the image data and conversation information and emotion recognition information extracted from the sound data.

For example, referring to FIG. 8, feature vectors related to human detection and tracking information, behavior recognition information, gesture recognition information, and facial expression recognition information may be extracted from the image data of the video clip 800 and used to measure the similarity. Also, feature vectors related to conversation information, background noise information, and emotion recognition information may be extracted from the sound data 820 of the video clip 800 and used to measure the similarity.

The memory 1030 stores ground truth information.

Also, the memory 1030 may support the above-described functions for social intelligence evaluation according to an embodiment of the present invention. Here, the memory 1030 may function as separate mass storage, and may include a control function for performing operations.

Meanwhile, the apparatus for evaluating social intelligence may include memory installed therein, thereby storing information in the apparatus. In an embodiment, the memory is a computer-readable recording medium. In an embodiment, the memory may be a volatile memory unit, and in another embodiment, the memory may be a nonvolatile memory unit. In an embodiment, the storage device is a computer-readable recording medium. In different embodiments, the storage device may include, for example, a hard-disk device, an optical disk device, or any other kind of mass storage.

Through the above-described apparatus for evaluating social intelligence, laymen may also evaluate human social intelligence in a short time more effectively than when using a method in which highly skilled experts observe the target to evaluated for a long time and acquire a result therefrom.

Also, it is possible to continuously observe and track the development process of the target to be evaluated regardless of where the target is.

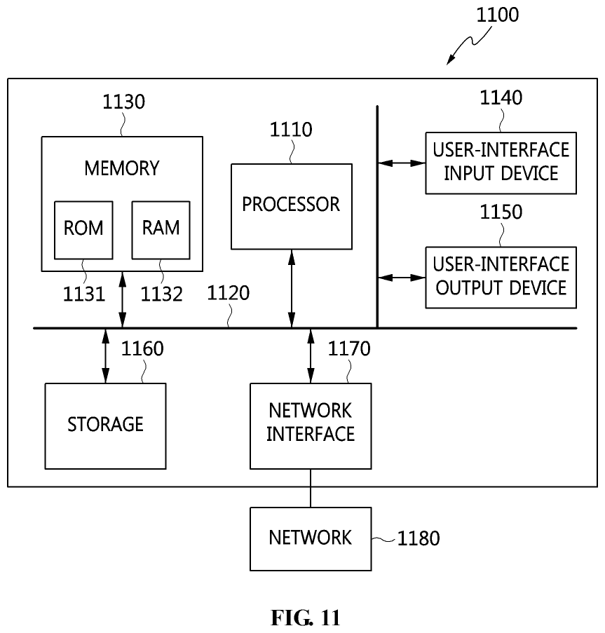

FIG. 11 is a view that shows a computer system according to an embodiment of the present invention.

Referring to FIG. 11, an embodiment of the present invention may be implemented in a computer system including a computer-readable recording medium. As illustrated in FIG. 11, the computer system 1100 may include one or more processors 1110, memory 1130, a user-interface input device 1140, a user-interface output device 1150, and storage 1160, which communicate with each other via a bus 1120. Also, the computer system 1100 may further include a network interface 1170 connected to a network 1180. The processor 1110 may be a central processing unit or a semiconductor device for executing processing instructions stored in the memory 1130 or the storage 1160. The memory 1130 and the storage 1160 may be various types of volatile or nonvolatile storage media. For example, the memory may include ROM 1131 or RAM 1132.

Accordingly, an embodiment of the present invention may be implemented as a nonvolatile computer-readable storage medium in which methods implemented using a computer or instructions executable in a computer are recorded. When the computer-readable instructions are executed by a processor, the computer-readable instructions may perform a method according to at least one aspect of the present invention.

According to the present invention, video data analysis and comparison methods may be applied to the evaluation of human social intelligence.

Also, the present invention may provide an automated evaluation tool for evaluating human social intelligence levels based on standardized evaluation criteria.

Also, the present invention may identify, in advance, people having a social intelligence problem and provide proper treatment and care services.

Also, the present invention may provide a method that enables laymen to evaluate human social intelligence in a short time more effectively than when using a method in which highly skilled experts observe an evaluation target for a long time and acquire a result therefrom.

Also, the present invention may provide a method for evaluating social intelligence through which the development of social intelligence of an evaluation target may be continuously evaluated and tracked regardless of the location of the evaluation target.

As described above, the method and apparatus for evaluating social intelligence according to the present invention are not limitedly applied to the configurations and operations of the above-described embodiments, but all or some of the embodiments may be selectively combined and configured, so that the embodiments may be modified in various ways.

What is claimed is:

1. A method for evaluating social intelligence, comprising:
segmenting, based on behavior recognition, an observation video sequence that captures social interaction behavior of a target to be evaluated, thereby creating multiple segmented video clips; and
calculating an evaluation score based on similarities between ground truth, which is created based on social interaction analysis, and the multiple segmented video clips, thereby evaluating social intelligence of the target.

2. The method of claim 1, wherein the ground truth corresponds to multiple verification video clips that are created by classifying an input video sequence pertaining to social interaction based on specific behavior items of an Evaluation of Social Interaction (ESI) scenario.

3. The method of claim 2, wherein evaluating the social intelligence of the target is configured to calculate the evaluation score by applying a score for each ESI item and a weight for specific behavior to each of the similarities, the score for each ESI item being set based on the ESI scenario, and the weight for specific behavior being set based on the specific behavior items.

4. The method of claim 2, wherein evaluating the social intelligence of the target comprises:
sequentially comparing the multiple segmented video clips with the multiple verification video clips and measuring the similarities through comparison of content of the video clips and comparison of a context of content that precedes and follows the video clips.

5. The method of claim 4, wherein the similarities are measured using cosine similarity between feature information extracted from the multiple segmented video clips and feature information extracted from the multiple verification video clips.

6. The method of claim 5, wherein the feature information is behavior recognition information and facial expression recognition information, which are extracted from image data, and conversation information and emotion recognition information, which are extracted from sound data.

7. The method of claim 1, wherein creating the multiple segmented video clips is configured to segment the observation video sequence into the multiple segmented video clips by performing behavior recognition based on at least one of an object detection function, an object-tracking function, and a gesture recognition function.

8. An apparatus for evaluating social intelligence, comprising:
a processor for creating multiple segmented video clips by segmenting, based on behavior recognition, an observation video sequence that captures social interaction behavior of a target to be evaluated, for calculating an evaluation score based on similarities between ground truth, which is created based on social interaction analysis, and the multiple segmented video clips, and for evaluating social intelligence of the target; and
memory for storing the ground truth.

9. The apparatus of claim 8, wherein the ground truth corresponds to multiple verification video clips that are created by classifying an input video sequence pertaining to social interaction based on specific behavior items of an Evaluation of Social Interaction (ESI) scenario.

10. The apparatus of claim 9, wherein the processor calculates the evaluation score by applying a score for each ESI item and a weight for specific behavior to each of the similarities, the score for each ESI item being set based on the ESI scenario, and the weight for specific behavior being set based on the specific behavior items.

11. The apparatus of claim 9, wherein the processor sequentially compares the multiple segmented video clips with the multiple verification video clips and measures the similarities through comparison of content of the video clips and comparison of a context of content that precedes and follows the video clips.

12. The apparatus of claim 11, wherein the similarities are measured using cosine similarity between feature information extracted from the multiple segmented video clips and feature information extracted from the multiple verification video clips.

13. The apparatus of claim 12, wherein the feature information is behavior recognition information and facial expression recognition information, which are extracted from image data, and conversation information and emotion recognition information, which are extracted from sound data.

14. The apparatus of claim 8, wherein the processor segments the observation video sequence into the multiple segmented video clips by performing behavior recognition based on at least one of an object detection function, an object-tracking function, and a gesture recognition function.
